(19) United States
(12) Patent Application Publication
(10) Pub. No.: US 2024/0428909 A1
(43) Pub. Date: Dec. 26, 2024

(54) EDGE-BASED MENTAL HEALTH ASSESSMENT

(71) Applicant: HEALTH RHYTHMS, INC., Long Island City, NY (US)

(72) Inventors: Mark J. Matthews, Dublin (IE); Denzil S.T. Ferreira, Oulu (FI)

(21) Appl. No.: 18/667,167

(22) Filed: May 17, 2024

Related U.S. Application Data
(60) Provisional application No. 63/509,212, filed on Jun. 20, 2023.

(51) Int. Cl. G16H 10/65; G16H 15/00; G16H 20/00; G16H 40/63; G16H 40/67

(52) U.S. Cl. CPC G16H 10/65; G16H 15/00; G16H 20/00; G16H 40/63; G16H 40/67

(57) ABSTRACT

In an embodiment, a computer-implemented method comprises using a computer system comprising one or more edge devices in a local computing environment, programming a first edge device to operate as an edge computing node, wherein the edge devices are configured to execute operations without requiring telecommunications to a remote server via an internet; and by the first edge device: identifying second edge devices being activated in the local computing environment, adding the first and second edge devices in an edge network, retrieving data from the second edge devices via the edge network, wherein the retrieved data comprises sensor data collected by the second edge devices, generating (at the first edge device) data summaries from the retrieved data, storing the data summaries in a storage associated with the first edge device, and presenting the data summaries via an application executing on the first edge device.

### BENEFIT CLAIM

This application claims the benefit under 35 U.S.C. § 119 (e) of provisional application 63/509,212, filed 20 Jun. 2023, the entire contents of which are hereby incorporated herein by reference for all purposes as if fully set forth herein.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright or rights whatsoever. ©2022-2023 Health Rhythms, Inc.

### TECHNICAL FIELD

One technical field of the present disclosure is resource-constrained computational devices, on-the-edge data processing, self-organizing local networks, and automated synthesis of database records.

### BACKGROUND

The approaches described in this section are approaches that could be pursued but not necessarily approaches that have been previously conceived or pursued. Therefore, unless otherwise indicated, it should not be assumed that any of the approaches described in this section qualify as prior art merely by virtue of their inclusion in this section.

The current state of the art on computational systems that model human behavior and mental health do so on computation-equipped remote machines and cloud premises, also commonly referred to as cloud computing in computer science. Such an approach has security, privacy, and latency implications when dealing with a privacy-aware clinical setting and personal data. Such systems can be inadequate for privacy-preserving systems that are compliant with user data privacy policies (HIPAA, GDPR, for example). The user may not be in control of their data once it leaves their device, nor is there always clarity on what such data is used for. Consequently, there is an acute need in the field of mental health diagnosis and treatment for on-the-edge computation and processing of data without reliance on remote machines and centralized computational infrastructures such as AWS, Azure, Google Cloud, and others.

Edge computing enables the computation of sensor and patient data within the boundaries of a delimited network, allowing communication with the Internet if necessary. Edge computing is related to mobile ad-hoc networks (MANETs), a resilient network concept developed for military and emergency response in the late 1970s (Abolhasan 2004). MANET's key characteristic is its dynamic network typology (Williams and Camp 2002). Just as MANETs, in edge computing, devices may exchange data via wireless communication protocols such as Bluetooth, and Wi-Fi when in proximity. The difference between MANET and edge computing is in the delegation aspect of computation: on the edge, nodes act entirely or semi-autonomously, capable of delegating computation to nearby devices and or performing data analysis on-device. On-the-edge, dynamic network typology provides optimal efficiency and low-latency connectivity. This patent envisions a conceptual framework where edge computing is utilized in personal healthcare.

### SUMMARY

The appended claims may serve as a summary of the invention.

### BRIEF DESCRIPTION OF THE DRAWINGS

In the drawings:

FIG. 1 is a state diagram that can be executed using multiple computing devices of

a self-organizing edge computing system in one embodiment.

FIG. 2A illustrates an example of an edge-computing architecture in which personal computing devices are used edge devices for health data.

FIG. 2B illustrates an example of the functional operation of an edge computing node in relation to other elements of the system of FIG. 2A.

FIG. 3 illustrates an example of a summary of daily noise data in an approach using a microphone of a mobile computing device to estimate noise exposure throughout the day.

FIG. 4 illustrates an example of a summary of time data for an individual.

FIG. 5 illustrates an example computing system that can be used in an embodiment.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

The text of this disclosure, in combination with the drawing figures, is intended to state in prose the algorithms that are necessary to program the computer to implement the claimed inventions at the same level of detail that is used by people of skill in the arts to which this disclosure pertains to communicate with one another concerning functions to be programmed, inputs, transformations, outputs and other aspects of programming. That is, the level of detail set forth in this disclosure is the same level of detail that persons of skill in the art normally use to communicate with one another to express algorithms to be programmed or the structure and function of programs to implement the inventions claimed herein.

### 1. General Overview

In one embodiment, the disclosure provides a resource-constrained computation infrastructure programmed to collect patient self-reported and/or passive sensor data from smartphones, wearable computing devices, and intelligent home components such as actionable light bulbs, weight scales, infrared sensors, refrigerators, cooking equipment, smart TVs, smart beds, cameras, Wi-fi routers, and other IoT household devices. Additionally or alternatively, non home-based devices can include sensors, and an embodiment can be programmed to collect data from those sensors. For example, banking data could be processed on the edge in a mobile/laptop environment as described before being transferred in an anonymized format to the cloud. In one embodiment, programmatic analysis of data collected from any of the foregoing devices is computed periodically in edge computing devices, independent of cloud or remote server processing, thereby enabling privacy-protective deployments because the data is not transmitted away from a collection point to a server of an enterprise or other entity. In one embodiment, external sources of data such as routers, air pollution, or weather stations may be integrated to add data about local conditions, situations, or other contexts. In one embodiment, these data sources can be used to more precisely appraise the individual level of the risk of a mental health state, condition, disorder, or event. For example, general high night light levels increase the risk of sleep disruption. In another embodiment, these external data sources can be used under program control to identify barriers and social determinant factors that need to be accounted for and, in some cases, addressed by the entity delivering care or interventions. As another example, external sources of data can use local computing resources to aggregate data for data summaries. For example, a smartwatch can use locally collected light sensor data and present a summary of the total time of daily sunlight exposure on the device, corresponding to a known mental health factor.

Computing devices that are logically located in edge positions can include devices that are in a physical or logical position that is separate from and/or disconnected from servers, data centers, and/or virtual compute instances of cloud computing systems. Such devices can be autonomous yet can share sensor data using a localized mesh network. For example, multiple smartphones can form a local mesh network. In this configuration, the local devices can operate in unison to aggregate and synthesize data to represent personalized insights into the behavioral data of one or more users, such as sleeping patterns, social activity, mobility, daily routines, and other mental or behavioral health indicators. Data summaries can be presented locally on a mobile device via an application or transmitted securely to a server computer and then presented remotely via a dashboard to a care provider; additionally or alternatively, data summaries can be stored and accessible via calls to an API of a server, enabling third-party entities to access the data summaries, subject to appropriate access, authentication, and authorization controls. This information can be used to personalize treatment via algorithmic interventions or to appraise individual mental health states and trends.

The disclosed techniques allow clinicians and other systems or algorithms to receive insight into the mental health of subjects or patients via data summaries that are obtained from patient devices, as the devices receive updates, changes, or replacements without having to offload sensitive personal data from an individual's device. The lifecycle of some wearable devices is estimated to be approximately six months, and smartphone ownership is about a year for some individuals. The software applications or “apps” on the devices usually are updated even more frequently. The present techniques provide an evolving sensing infrastructure where novel sensing devices and processing algorithms can be added without disrupting data collection and analysis in mental health patient care. In one embodiment, the sensing infrastructure can interoperate with multiple simultaneous sensing sources for specific variables at the individual and group levels. For example, the system can communicate, in relation to a single participating individual, with a smartphone of the individual to estimate sleep and a wearable device and/or a smart bed that also reports sleep data. Data from a smart bed may have greater accuracy and precision than data from a smartphone concerning sleep. Another individual may only use a smartphone or some other source. Embodiments can be programmed to assess behavioral and mental health regardless of the data source and account for source variability.

In an embodiment, the disclosure provides a computer-implemented method executed at a computer system and comprising: using the computer system comprising a plurality of edge devices in a local computing environment, programming a first edge device of the plurality of edge devices to operate as an edge computing node, wherein the plurality of edge devices are configured to execute operations without requiring telecommunications to a remote server via an internet; by the first edge device of the computer system, identifying one or more second edge devices of the plurality of edge devices being activated in the local computing environment; by the first edge device of the computer system, adding the first and second edge devices in an edge network; by the first edge device of the computer system, retrieving data from the one or more second edge devices via the edge network, wherein the retrieved data comprises sensor data collected by the one or more second edge devices; by the first edge device of the computer system, generating one or more data summaries from the retrieved data based on one or more models; by the first edge device of the computer system, storing the one or more data summaries in a storage associated with the first edge device; and by the first edge device of the computer system, presenting the one or more data summaries via an application executing on the first edge device.

In some embodiments, identifying the one or more second edge devices is based on one or more of a network protocol, a proximity protocol, or a device discovery protocol.

In some embodiments, the computer-implemented method executed at the computer system further comprises: determining the first edge device to be the edge computing node using one or more voting protocols based on metadata associated with the plurality of edge devices, the metadata comprising one or more of a device type, a CPU type, or an amount of summary.

In some embodiments, the edge network comprises a local mesh network. Adding each of the first and second edge devices in the edge network comprises one or more of: supplying a session key for encrypted local communication; providing one or more LAN access credentials; providing one or more Wi-Fi router access credentials; or adding the edge device to a list of devices in the edge network that is digitally stored at the edge computing node.

In some embodiments, the computer-implemented method executed at the computer system further comprises: by the first edge device of the computer system, transmitting one or more of a data summary or an update to a cloud computing system. Transmitting one or more of the data summary or the update to the cloud computing system is based on a message bus or an event bus.

In some embodiments, retrieving data from the one or more second edge devices via the edge network comprises: by the first edge device of the computer system, determining, a respective device type for each of the one or more second edge devices; and by the first edge device of the computer system, transmitting, to each of the one or more second edge devices, a respective request for data formatted based on the respective device type, wherein the respective request for data is configured for causing the corresponding second edge device to respond with the data requested. The request for data comprises one or more of a single discrete command, a single discrete ping, or an application programming interface (API) call.

In some embodiments, the computer-implemented method executed at the computer system further comprises: by the first edge device of the computer system, de-identifying the one or more data summaries.

In some embodiments, the computer-implemented method executed at the computer system further comprises: by the first edge device of the computer system, encrypting the one or more data summaries.

In some embodiments, the storage is based on one or more of an in-app memory, an on-device memory, or a network-attached storage. All storage devices can use encrypted storage techniques.

In some embodiments, the one or more data summaries comprise one or more mental health indicators comprising one or more of a sleeping pattern, a social activity, a mobility, a level of technology use, a level of light exposure, data concerning diet, or data concerning a daily routine.

In some embodiments, the computer-implemented method executed at the computer system further comprises: by the computer system, generating, based on the one or more data summaries, a personalized treatment via algorithmic interventions.

In some embodiments, the computer-implemented method executed at the computer system further comprises: by the computer system, generating, based on the one or more data summaries, an appraisal of mental health states and trends.

Certain embodiments describe the use of a first edge device and second edge device, or refer to edge networks consisting of multiple devices. However, one embodiment also can use a single edge device. For example, a single edge device hosting an app or capable of calling an API via a mobile browser, the single edge device could access extensive data sourced from other devices and useful in other aspects of the processes and systems of the disclosure. For example, a single edge device could be programmed to call functions of the Apple Health API to access Apple Watch data and/or retrospective physical activity dynamics computed from smartphone sensors or using other APIs. Calls to other services could obtain data summarizing vocal dynamics, time at home, apps used, and other aspects of user activity. Some of these sources require other devices, but embodiments of the present disclosure do not need to interface with them provided relevant OS or software services are available to serve the data of interest.

### 2. Structural & Functional Overview

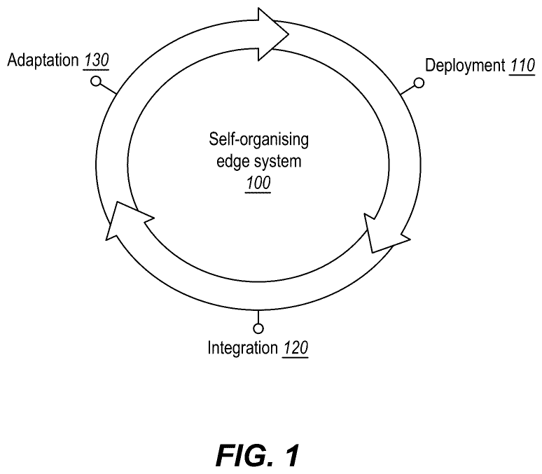

FIG. 1 is a state diagram that can be executed using multiple computing devices of a self-organizing edge computing system in one embodiment. FIG. 1, and the other drawing figures and all of the description and claims in this disclosure, are intended to present, disclose and claim a technical system and technical methods in which specially programmed computers, using a special-purpose distributed computer system design, execute functions that have not been available before to provide a practical application of computing technology to the problem of automatically collecting and analyzing sensor data and reporting results for use in supporting interventions or care. In this manner, the disclosure presents a technical solution to a technical problem, and any interpretation of the disclosure or claims to cover any judicial exception to patent eligibility, such as an abstract idea, mental process, method of organizing human activity, or mathematical algorithm, has no support in this disclosure and is erroneous.

In the example of FIG. 1, a plurality of computing devices form a self-organizing edge system 100 by asynchronously executing functions of three stages, in which each stage transitions to a successive stage in one direction. In the first stage 110, termed deployment, sensing devices or algorithms are introduced into the system; for example, an end user might power up and join to a local area network a smartphone, tablet computer, or other computing device with sensor capability that has not been used in the system 100 previously.

In the second stage 120, termed integration, the new devices or algorithms are connected to other data processing components. For example, the end user might launch applications or apps on the smartphone, tablet computer, or other computing device; the new devices or algorithms may call APIs or execute specified protocols with networked communication among one or more external servers, resources, or endpoints, to conduct registration or integration; during execution, the apps on those devices could be programmed to establish programmatic connections via the LAN to other devices in the system 100, or via wide area networks or the public Internet to servers of the system. Thus, as the end user acquires new devices, the devices can become additional sources of data. They can be integrated into their edge-based mental health by means of API integration or on-the-edge device deployment. The API integration of the device could be provided by the manufacturer of such device, e.g., Apple Health, Health Connect APIs. On-device integration deployment can be achieved via software deployed directly to the device, e.g., a smartwatch application, which computes and aggregates data and presents summaries on-device. The data can also be shared on a LAN using multicast for data subscribers.

In the third stage 130, termed adaptation, personalized data summaries are calculated and presented or transmitted.

The terms first, second, and third, and the labels 110-130, are used merely to distinguish different stages or states, and other labels or terms could be used in other embodiments.

The impact of FIG. 1 is that the present disclosure contemplates a resilient computing system in which existing computing devices with data describing one another can automatically integrate and adapt to the introduction of a new device into the system, including changing the form and content of data summaries or reports based on data that is newly available from the newly introduced device. For example, for assessing bedtime and wake time, sleep duration, and sleep interruptions, a particular user could initially use a smartphone-based estimator of the foregoing variables. The same user later could connect to the network a wearable device that has close proximity to the body and, therefore, less noise for the same variables. Embodiments can be programmed to give these devices priority over coarser sources. The edge network, once it receives new data, even for retrospective days, can update a database table, in-memory table, or other digitally stored, ordered list of the priority of data sources. The priority order can be updated on a daily basis. If days are missing for the wearable device, then the next best trusted source can used. If there is no passive source, then a self-assessment could be prompted.

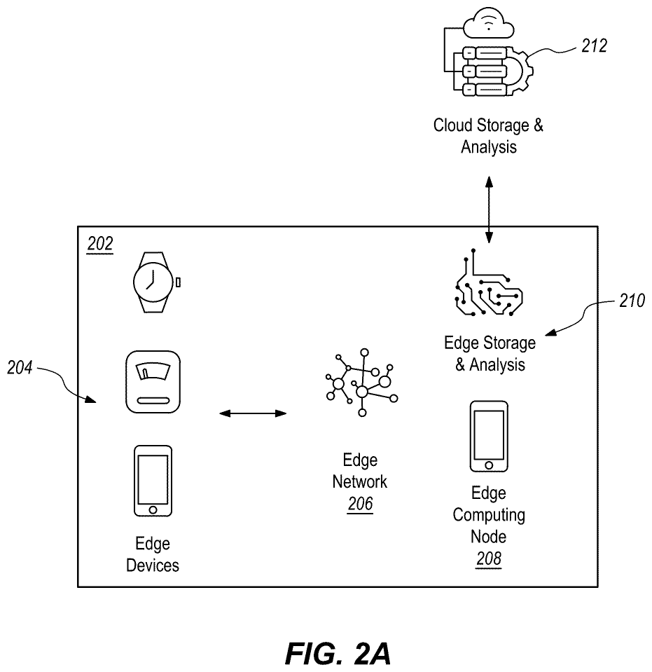

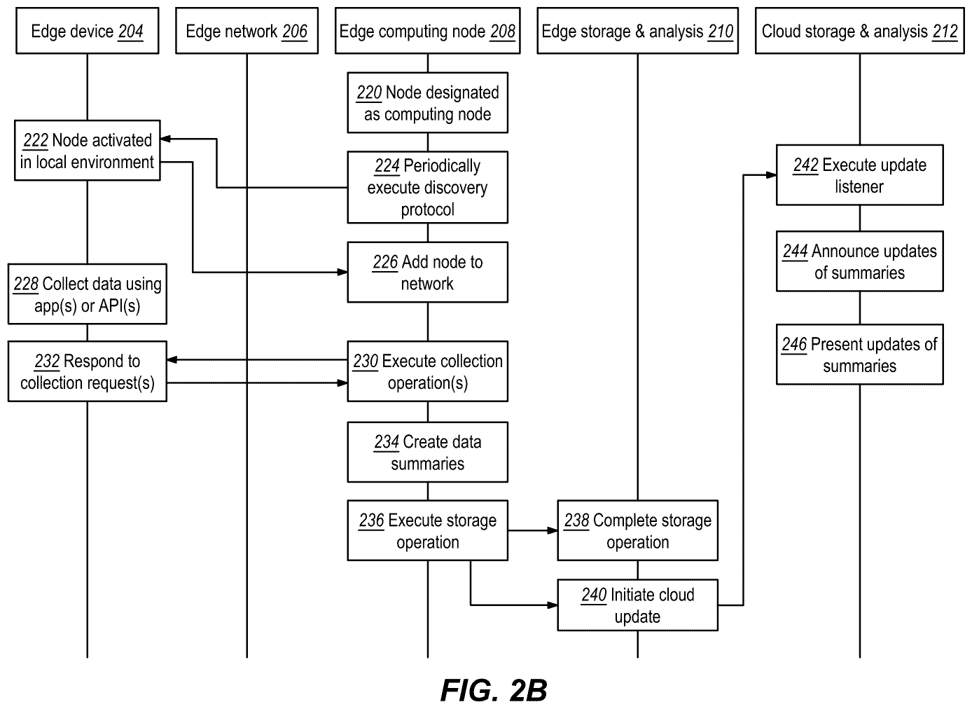

FIG. 2A illustrates an example of an edge-computing architecture in the context of personal devices as edge devices for health data. In this context, the term “edge computing” refers to executing machine-implemented operations using a plurality of computer devices that do not require telecommunications to a server via an internetwork or the public internet. For example, selected elements of FIG. 2A could be associated with a home, office, or other discrete location and could rely on local area networks, short-distance networks, or near-field communications rather than always conducting long-distance telecommunications to a remote server computer.

In one embodiment, an edge system 202 comprises a plurality of different edge devices 204, each of which may be associated with the same individual user or with different users who are near one another or become proximate to one another periodically by moving. Examples of edge devices include a smartwatch, a smart scale, a smartphone, tablet, or other mobile computing device or Internet of Things (IoT) device. Each of the edge devices 204 hosts or executes an edge processing mobile application or app that is capable of discovering, logically linking to, and forming an edge network 206 comprising that edge device 204 and other nearby edge devices 204. The edge network 206 can comprise a localized mesh network.

In an embodiment, one of the edge devices 204 is programmed to operate as an edge computing node 208. Using peer-to-peer and device APIs of the edge network 206, the edge computing node 208 is programmed to retrieve data from the other edge devices 204, analyze the data, and store the data as part of edge storage and analysis operations 210. In an embodiment, the edge computing node 208 comprises digital data storage for storing data summaries that the edge computing node 208 creates as part of edge storage and analysis operations 210; in other embodiments, the edge computing node 208 directs storage to a local storage device that is attached to the edge network 206.

The edge computing node 208 may communicate with external remote services and storage such as cloud storage and analysis 212, but the other edge devices 204 do not. For example, the edge computing node 208 may be programmed to periodically report the data summaries to cloud storage and analysis 212, whereas the edge devices 204 do not store data specifying network addresses for the cloud storage and analysis, so the edge devices 204 cannot directly contact that element. In an embodiment, the edge computing node 208 transmits updates to cloud storage and analysis 212 using a digitally stored schedule; for example, updates can be transmitted every hour, every four hours, once daily, or at other intervals. In one embodiment, cloud storage and analysis 212 comprises one or more virtual compute instances and/or virtual storage instances of a cloud computing center or private data center.

In an embodiment, a new mobile computing device may be acquired by the user, or an existing mobile computing device may install or update a local healthcare app. A first local device, such as a smartphone or software, is programmed to detect and integrate such a mobile computing device as a sensor to acquire a new set of data from that mobile computing device or app. The system then programmatically executes functions to adapt by incorporating the new data into analysis and summarization in the edge computing node 208, without cloud or remote resources. In an embodiment, a new algorithm may be deployed remotely via a software update on a device, which can integrate the processing of sensor data from various sensors and adapt it for a novel data summary. The specific process flows or programming that the new algorithm implements are not critical; instead, the importance of this step is that whenever a new app or application is received on a device via a software update, the new app or application can execute to acquire data from the sensors and execute functions not previously implemented.

As new devices are introduced, disabled, become faulty, or are replaced, edge devices 204 are programmed to determine the appearance and disappearance of data sources. For example, network and proximity protocols such as Bluetooth, Zigbee, closed and/or proprietary wireless device protocols, another ad hoc device discovery protocol, and/or Wi-Fi device discovery can be used to identify new edge devices 204 that have appeared in the system 202 and execute adaptation functions in response. The disappearance of a data source can be determined if a device is no longer available, that is, a ping or discovery protocol no longer can find the device on a local network. In an edge network, nodes perform a heartbeat (i.e., ping) periodically. If a node fails to update its routing, it may be unavailable temporarily, or permanently. In an embodiment edge computing node 208 is programmed to use a temporal filter to remove sources that have been unavailable for an extended period (weeks, months, or years, in various embodiments. On a nearby protocol, the edge computing node 208 can be programmed to execute periodic scans; for example, Bluetooth scan results and Bluetooth LE advertising packets can be used to determine the availability of a data source.

In an embodiment, a new data model or process of data analysis may be deployed to an existing mobile computing device via an update or local healthcare app. The device may advertise the availability of the model and/or start processing on-the-edge data to create novel data summaries or abstractions. For example, a new pre-trained machine learning model using OpenCV, Tensorflow, or other machine learning systems could be deployed to a smartphone and executed in an inference stage to detect and infer sentiment or mood from image data representing facial expressions. In another embodiment, a new algorithm to estimate sleep time from microphone sound analysis could be deployed and executed on the mobile device and a summary could be generated offline. The processing could occur locally on-device, primarily for privacy, or could occur in an on-the-edge device to improve performance.

### 3. Implementation Example—Software Overview

In one embodiment, the data analyses are routinely computed on the edge computing node 208 and/or edge devices 204, independent of cloud or remote server processing. Such devices are autonomous and can share sensor data employing a localized mesh network, working in unison to aggregate, synthesize, and represent personalized insight into one's behavioral data, such as sleeping patterns, social activity, mobility, daily routines, and other mental health indicators. Data summaries can then be presented locally on the edge computing node 208 and/or edge devices 204 via a purpose-built application and/or remotely to a care provider.

FIG. 2B illustrates an example of selected functional operations of elements of the system of FIG. 2A. FIG. 2B presumes that at least one edge device 204 is present in a local computing environment and that at least one server computer functioning to provide cloud storage and analysis 212 has been deployed in an addressable, routable network via a LAN, WAN, campus network, or internetwork. In an embodiment, at block 220, the edge computing node 208 is programmed to designate itself or receive a designation as an edge computing node 208. Block 220 can comprise executing a voting protocol or election protocol based on metadata that one edge device 204 collects from all other edge devices under stored program control, using an app on that edge device 204. For example, edge devices 204 can be programmed to collect data describing the type of device or CPU, amount of memory, or other attributes on which a decision can be based to vote for or elect a node to serve as the edge computing node 208. For example, one of the edge devices 204 having the fastest processor or greatest amount of memory could be elected. Multiple edge computing nodes 208 can be used in some embodiments. Depending on the computational capacity of each node among multiple edge computing nodes 208, data analysis can be performed concurrently between them to speed up computation.

At block 222, one or more edge devices 204 are introduced to the local environment and powered up, updated, or otherwise activated for use.

At block 224, the edge computing node 208 periodically executes a discovery protocol to identify other edge devices 204, including one or more new edge devices 204 that have been activated in the local environment, as shown at block 222. For example, an embodiment can be programmed to execute block 224 by reading a master or wildcard list of sources of edge devices and data sources, and polling each device or source on the list to determine whether they are active. Alternatively, block 224 can comprise reading a list of specific devices that are expected to be in the network, and successively attempting to communicate with each device on the list over the network. On a mesh network topology, a heartbeat can be used to inform and announce to the network the availability of a node. Master nodes can keep track of the routes to nearby edge devices and manage this routing table. ARP (Address Resolution Protocol) requests can be used to discover networked devices. Nearby scans can be used for Bluetooth & BLE advertising packets can be used for device discovery.

At block 226, the edge computing node 208 adds the newly activated edge device(s) 204 to the edge network 206. In one embodiment, adding an edge device 204 to the edge network 206 can comprise supplying a session key for encrypted local communication, providing LAN access credentials, Wi-Fi router access credentials, and/or adding the edge device 204 to a list of devices in the edge network 206 that is digitally stored at the edge computing node 208.

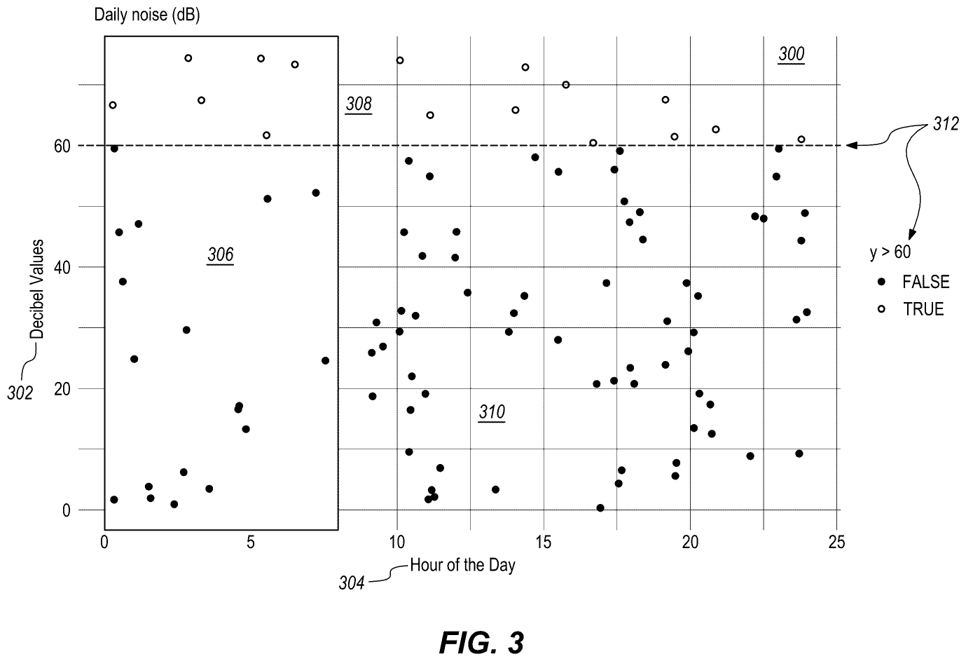

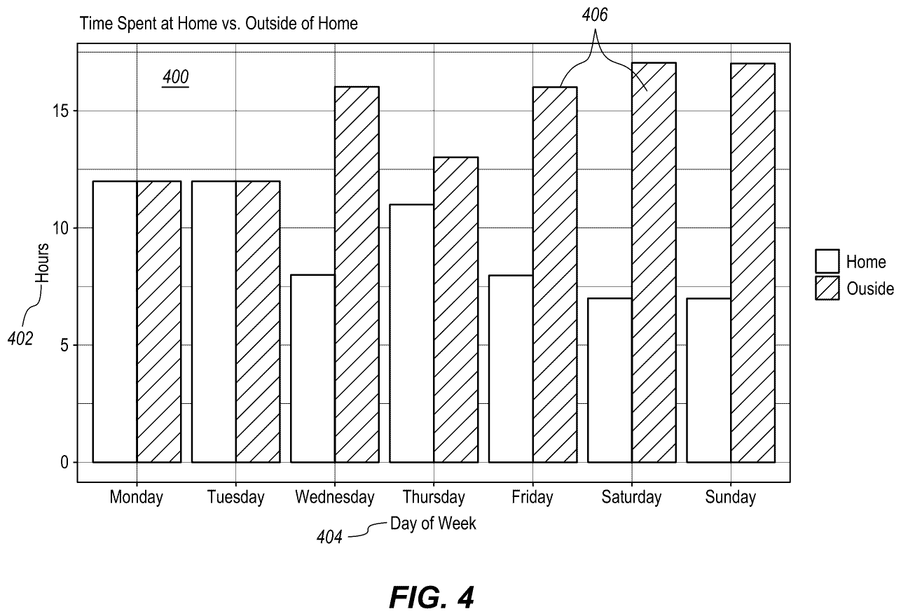

At block 228, each of the edge devices 204 in the edge network 206 asynchronously collects data using one or more APIs associated with networked servers, device-specific applications, or apps. The form, functions, and programming of each of the apps will vary depending upon which kind of edge device 204 the apps execute on. The term “app,” in this context, includes smartphone or smartwatch apps that can be obtained from app stores, as well as a single program, set of instructions, firmware, or hardware such as an ASIC or FPGA that implements one or more functions. For example, an app executing in a digital scale could merely record and store a weight value and a timestamp for the weight value; other apps could be much more complex and could provide data like that described herein for FIG. 3, FIG. 4.

At block 230, edge computing node 208 executes one or more collection operations by polling, querying, or otherwise collecting data from one or more edge devices 204. The collection operations can be scheduled or continuous. The data collection operations can be programmed to consumer retrospective data. For example, if a user signs up to the network, then block 230 can be programmed to request and retrieve any available retrospective data to assess the individual's current state and past patterns of behavior. In one embodiment, the edge computing node 208 continuously executes a round-robin scheme to successively select and contact all the edge devices 204 that are known to participate in the edge network 206. In this context, executing a round-robin scheme means that the edge computing node 208 is programmed to successively select the next edge device 204 and contact that device, then move on to the next known edge device for contact. Specific collection operations, messages, calls, or requests can vary, depending upon the type of the target edge device 204. For example, some edge devices 204 may require only a single discrete command or ping to cause responding, at block 232, to the collection request and others may use a more complex API that requires a programmatic call with an access key, function identifier, and parameters, communicated programmatically using a parameterized HTTP request, or app-specific protocol. Blocks 230, 232 can iterate repeatedly as the edge computing node 208 collects data from one or more edge devices 204.

At block 234, edge computing node 208 is programmed to create one or more data summaries from the data that was collected at blocks 230, 232. Data summaries at block 234 can be de-identified and/or encrypted to increase the privacy of users of edge devices 204. Examples of the presentation of data summaries are described herein in connection with FIG. 3 and FIG. 4. A data summary can comprise a de-identified aggregation of multiple data values into one or more aggregated data values or can comprise data representing a table, chart, graph, or other visualization or calculation that has been derived from the original data. For example, a data summary could be a set of median values or mean values with or without standard deviation for each hour of the day, calculated based on a much larger set of raw data values that were collected multiple times during each hour. Each data summary can be formed by executing a transformation and/or inference algorithm based on the original data.

At block 236, the edge computing node 208 is programmed to execute a storage operation by transmitting one or more of the data summaries to edge storage and analysis operations 210 with a request or message containing instructions to store the data summaries. As noted earlier, storage can occur in in-app memory of the edge computing node 208, other on-device memory, and/or network-attached storage. At block 238, the selected storage device completes the requested storage operation; for example, a completion, success, or acknowledgment message can be returned to edge computing node 208.

At block 240, the edge computing node 208 and/or the edge storage and analysis operations 210 initiate a cloud update, comprising transmitting one or more data summaries to the cloud storage and analysis 212. In some embodiments, a virtual compute instance of a cloud computing center executes an update listener program or is programmed to await calls from the cloud update operation of block 240. A message bus or event bus can be used for inter-process communication between edge storage and analysis operations 210 and cloud storage and analysis 212. Block 242 can include operations to store the data summaries in virtual storage or other server-side storage, including encryption or further anonymization operations. Blocks 240, 242 can use a request-response protocol that iterates repeatedly until the edge computing node 208 and/or edge storage and analysis operations 210 have delivered all available updates to cloud storage and analysis 212. Updates can include version marking and/or timestamping to enable the cloud storage and analysis 212 to recover if a loss of connectivity occurs during updating operations.

In block 244, the cloud storage and analysis 212 can be programmed to announce one or more updates of data summaries via notifications, alerts, or other messages to a clinical care team, healthcare provider, family member, or another recipient. Additionally, or alternatively, block 244 can comprise updating a system or server that is programmatically accessible via an API, which in turn updates another system with which the present system is associated as a third-party service. For example, the edge network of the present disclosure could provide a continuous assessment of mental health as a component, function, or federated application of a diabetes management software system run by a different entity.

At block 246, the cloud storage and analysis 212 can be programmed to present updates of the data summaries to a clinical care team, healthcare provider, family member, or another recipient, using a presentation layer such as dynamic HTML and HTTPS to present web pages, a dashboard, or in-app updates when the recipients are using mobile computing devices.

FIG. 3 illustrates an example of a data summary that can be computed, stored, and presented, in one embodiment. The example presents a data summary of daily noise in decibels (dB) for a particular user. In an embodiment, a smartphone app, API, or SDK is programmed to access and use a microphone of the smartphone to collect data representing noise exposure throughout the day. The SDK can be embedded within another application, and the API could be programmatically accessible at an edge location to request and take in sensor data and process the data. Most users charge their devices overnight; thus, microphone sensor sensing can be continuous. Other devices could include a nightstand with a smart lightbulb, smartphone display, or alarm clock. Based on the microphone data, the smartphone app is programmed to calculate a summary of the maximum decibel level of all sound observed in a discrete period and store a data table of the decibel level values and corresponding time values in local app storage. The data in the table can then be presented to the user or transmitted to a care provider. A presentation to a user can use a visual representation in the smartphone display and/or a natural language summary delivered within a user interface or via a chat agent. The data can be transmitted to a care provider for presentation in a dashboard.

In the example of FIG. 3, a scatter plot 300 comprises a plurality of dots, circles, or other markers, each of which represents a discrete data point that the smartphone app derived from the audio measurements described in the preceding paragraph. In plot 300, a vertical axis 302 indicates the magnitude of decibel values of the data points, and a horizontal axis 304 specifies an hour of the day in which the data was detected. A shaded area 306 identifies points that the app recorded during nighttime, determined by the last interaction (sleep start) and first interaction (sleep end) with the device. An upper region 308 and a lower region 310 are defined using a threshold level 312, which can correspond to a particular noise level of interest, which is 60 decibels in the example of FIG. 3. In one example summary, data points in the upper region 308 and within the shaded area 306 can represent instances of likely sleep interruption or disturbance, arising from snoring, from ambient noise, or from other events. In this manner, plot 300 and similar data summaries can inform the patient (in an intervention study) and/or only the caregiver (observational study) of sleep disturbances.

FIG. 4 illustrates a second example of a data summary that can be computed, stored, and presented in one embodiment. The example of FIG. 4 comprises a bar chart 400 titled “Time Spent at Home vs Outside of Home” and represents an aggregate number of hours that a particular device, corresponding to a particular user, subject, or patient, was in a home or outside a home. A vertical axis 402 represents the magnitude of hours per day, and a horizontal axis 404 represents the days of the week; a plurality of bars 406 specify magnitude values based on collected data for time spent in the home and outside the home.

In one embodiment, software using DD-WRT (https://dd-wrt.com/) is installed on a patient's home router. During operation, the DD-WRT software executes to detect connections of a mobile computing device of the patient to the router. A period of time during which the mobile computing device is connected to the home router is recorded as time at home. When the mobile computing device is not discoverable on the local network, then the user is outside the home.

Additionally or alternatively, in an embodiment, detecting time at home could be inferred via the user's charging routine (Ferreira, Dey, and Kostakos 2011), considering nearby access points as a fingerprint for home versus outside of the home. This information could be summarized and presented to the user via an app or could be invisible to the end-user and presented to caregivers, or served via an API for third-party services to use, subject to the end-user's permission and consent. This summary can be a proxy for social isolation, anxiety, and/or sickness (Asare et al. 2021). In this example, the patient spends the most time outdoors on the weekends. This approach is privacy-preserving, as it does not reveal nor depend on location tracking.

### 4. Implementation Example—Hardware Overview

According to one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques or may include digital electronic devices such as a wearable that is programmed to perform the techniques or may include at least one general-purpose software processor programmed to perform the techniques pursuant to program instructions in firmware, memory, other storage, or a combination. Such computing devices may also combine custom algorithmic logic with custom programming to accomplish the described techniques. The computing devices may be handheld devices, mobile computing devices, wearable devices, body-mounted or implantable devices, smartphones, smart appliances, internetworking devices, any other electronic device that incorporates program logic to implement the described techniques, one or more virtual computing machines or instances in a data center, and/or a network of server computers and/or personal computers.

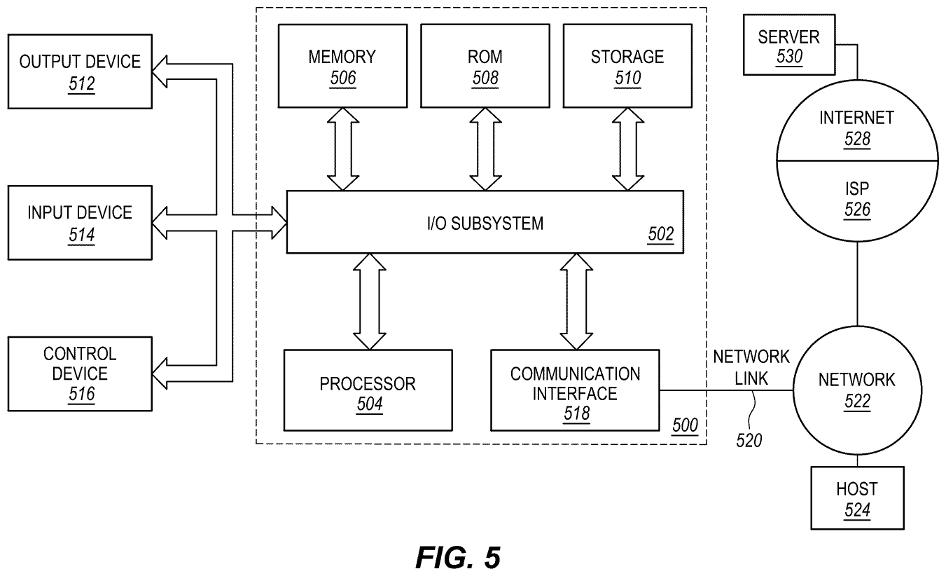

FIG. 5 is a block diagram that illustrates an example computer system with which an embodiment may be implemented. In the example of FIG. 5, a computer system 500 and instructions for implementing the disclosed technologies in hardware, software, or a combination of hardware and software are represented schematically, for example, as boxes and circles, at the same level of detail that is commonly used by persons of ordinary skill in the art to which this disclosure pertains for communicating about computer architecture and computer systems implementations.

Computer system 500 includes an input/output (I/O) subsystem 502, which may include a bus and/or other communication mechanism(s) for communicating information and/or instructions between the components of the computer system 500 over electronic signal paths. The I/O subsystem 502 may include an I/O controller, a memory controller, and at least one I/O port. The electronic signal paths are represented schematically in the drawings, for example, as lines, unidirectional arrows, or bidirectional arrows.

At least one central processing unit, such as a hardware processor 504, is coupled to I/O subsystem 502 for processing information and instructions. Hardware processor 504 may include, for example, a general-purpose microprocessor or microcontroller and/or a special-purpose microprocessor such as an embedded system, a graphics processing unit (GPU), a digital signal processor, or an ARM processor. Processor 504 may comprise an integrated arithmetic logic unit (ALU) or may be coupled to a separate ALU.

Computer system 500 includes one or more units of memory 506, such as a main memory, which is coupled to I/O subsystem 502 for electronically digitally storing data and instructions to be executed by processor 504. Memory 506 may include volatile memory such as various forms of random-access memory (RAM) or other dynamic storage device. Memory 506 may also be used for storing temporary variables or other intermediate information during the execution of instructions to be executed by processor 504. Such instructions, when stored in non-transitory computer-readable storage media accessible to processor 504, can render computer system 500 into a special-purpose machine that is customized to perform the operations specified in the instructions.

Computer system 500 further includes non-volatile memory such as read-only memory (ROM) 508 or other static storage devices coupled to I/O subsystem 502 for storing information and instructions for processor 504. The ROM 508 may include various forms of programmable ROM (PROM), such as erasable PROM (EPROM) or electrically erasable PROM (EEPROM). A unit of persistent storage 510 may include various forms of non-volatile RAM (NVRAM), such as FLASH memory, solid-state storage, magnetic disk, or optical disks such as CD-ROM or DVD-ROM and may be coupled to I/O subsystem 502 for storing information and instructions. Storage 510 is an example of a non-transitory computer-readable medium that may be used to store instructions and data, which, when executed by the processor 504, cause performing computer-implemented methods to execute the techniques herein.

The instructions in memory 506, ROM 508, or storage 510 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming, or other functions; data protocol instructions or stacks to implement TCP/IP, HTTP, or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server, or web client. The instructions may be organized as a presentation layer, application layer, and data storage layer, such as a relational database system using structured query language (SQL) or no SQL, an object store, a graph database, a flat file system, or other data storage.

Computer system 500 may be coupled via I/O subsystem 502 to at least one output device 512. In one embodiment, output device 512 is a digital computer display. Examples of a display that may be used in various embodiments include a touchscreen display, a light-emitting diode (LED) display, a liquid crystal display (LCD), or an e-paper display. Computer system 500 may include other type(s) of output devices 512, alternatively or in addition to a display device. Examples of other output devices 512 include printers, ticket printers, plotters, projectors, sound cards or video cards, speakers, buzzers or piezoelectric devices or other audible devices, lamps or LED or LCD indicators, haptic devices, actuators or servos.

At least one input device 514 is coupled to I/O subsystem 502 for communicating signals, data, command selections, or gestures to processor 504. Examples of input devices 514 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (IMU) sensors and/or various types of transceivers such as wireless, such as cellular or Wi-Fi, radio frequency (RF) or infrared (IR) transceivers and Global Positioning System (GPS) transceivers.

Another type of input device is a control device 516, which may perform cursor control or other automated control functions such as navigation in a graphical interface on a display screen, alternatively or in addition to input functions. The control device 516 may be a touchpad, a mouse, a trackball, or cursor direction keys for communicating direction information and command selections to processor 504 and for controlling cursor movement on an output device 512 such as a display. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism, or other type of control device. An input device 514 may include a combination of multiple different input devices, such as a video camera and a depth sensor.

In another embodiment, computer system 500 may comprise an Internet of Things (IoT) device in which one or more of the output device 512, input device 514, and control device 516 are omitted. Or, in such an embodiment, the input device 514 may comprise one or more cameras, motion detectors, thermometers, microphones, seismic detectors, other sensors or detectors, measurement devices or encoders, and the output device 512 may comprise a special-purpose display such as a single-line LED or LCD display, one or more indicators, a display panel, a meter, a valve, a solenoid, an actuator or a servo.

When computer system 500 is a mobile computing device, input device 514 may comprise a global positioning system (GPS) receiver coupled to a GPS module that is capable of triangulating to a plurality of GPS satellites, determining and generating geo-location or position data such as latitude-longitude values for a geophysical location of the computer system 500. Output device 512 may include hardware, software, firmware, and interfaces for generating position reporting packets, notifications, pulse or heartbeat signals, or other recurring data transmissions that specify a position of the computer system 500, alone or in combination with other application-specific data, directed toward host computer 524 or server computer 530.

Computer system 500 may implement the techniques described herein using customized hard-wired logic, at least one ASIC or FPGA, firmware, and/or program instructions or logic which, when loaded and used or executed in combination with the computer system, causes or programs the computer system to operate as a special-purpose machine. According to one embodiment, the techniques herein are performed by computer system 500 in response to processor 504 executing at least one sequence of at least one instruction contained in main memory 506. Such instructions may be read into main memory 506 from another storage medium, such as storage 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory computer-readable storage media that store data and/or instructions that cause a machine to operate in a specific fashion. Such storage media may comprise non-volatile media. Non-volatile media includes, for example, optical or magnetic disks, such as storage 510 whereas volatile media includes dynamic memory, such as memory 506. Common forms of non-transitory computer-readable storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire, and fiber optics, including the wires that comprise a bus of I/O subsystem 502. Transmission media can also take the form of acoustic or light waves, such as those generated during radio-wave and infrared data communications.

Various forms of media may be involved in carrying at least one sequence of at least one instruction to processor 504 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 500 can receive the data on the communication link and convert the data to a format that can be read by computer system 500. For instance, a receiver such as a radio frequency antenna or an infrared detector can receive the data carried in a wireless or optical signal, and appropriate circuitry can provide the data to I/O subsystem 502, such as placing the data on a bus. I/O subsystem 502 carries the data to memory 506, from which processor 504 retrieves and executes the instructions. The instructions received by memory 506 may optionally be stored on storage 510 either before or after execution by processor 504.

Computer system 500 also includes a communication interface 518 coupled to I/O subsystem 502 or a bus. Communication interface 518 provides a two-way data communication coupling to network link(s) 520 that are directly or indirectly connected to at least one communication network, such as a network 522 or a public or private cloud on the Internet. For example, communication interface 518 may be an Ethernet networking interface, integrated-services digital network (ISDN) card, cable modem, satellite modem, or a modem to provide a data communication connection to a corresponding type of communications line, for example, an Ethernet cable or a metal cable of any kind or a fiber-optic line or a telephone line. Network 522 broadly represents a local area network (LAN), wide-area network (WAN), campus network, internetwork, or any combination thereof. Communication interface 518 may comprise a LAN card to provide a data communication connection to a compatible LAN, a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 518 sends and receives electrical, electromagnetic, or optical signals over signal paths that carry digital data streams representing various types of information.

Network link 520 typically provides electrical, electromagnetic, or optical data communication directly or through at least one network to other data devices, using, for example, satellite, cellular, Wi-Fi, or BLUETOOTH technology. For example, network link 520 may provide a connection through network 522 to a host computer 524.

Furthermore, network link 520 may provide a connection through network 522 or to other computing devices via internetworking devices and/or computers that are operated by an Internet Service Provider (ISP) 526. ISP 526 provides data communication services through a worldwide packet data communication network represented as Internet 528. A server computer 530 may be coupled to Internet 528. Server computer 530 broadly represents any computer, data center, virtual machine, or virtual computing instance with or without a hypervisor or computer executing a containerized program system such as DOCKER or KUBERNETES. Server computer 530 may represent an electronic digital service that is implemented using more than one computer or instance and that is accessed and used by transmitting web services requests, uniform resource locator (URL) strings with parameters in HTTP payloads, API calls, app services calls, or other service calls. Computer system 500 and server computer 530 may form elements of a distributed computing system that includes other computers, a processing cluster, a server farm, or other organizations of computers that cooperate to perform tasks or execute applications or services. Server computer 530 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming, or other functions; data protocol instructions or stacks to implement TCP/IP, HTTP, or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server computer 530 may comprise a web application server that hosts a presentation layer, application layer, and data storage layer such as a relational database system using a structured query language (SQL) or no SQL, an object store, a graph database, a flat file system, or other data storage.

Computer system 500 can send messages and receive data and instructions, including program code, through the network(s), network link 520, and communication interface 518. In the Internet example, a server computer 530 might transmit a requested code for an application program through Internet 528, ISP 526, local network 522, and communication interface 518. The received code may be executed by processor 504 as it is received, and/or stored in storage 510, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 504. While each processor 504 or core of the processor executes a single task at a time, computer system 500 may be programmed to implement multitasking to allow each processor to switch between tasks that are being executed without having to wait for each task to finish. In an embodiment, switches may be performed when tasks perform input/output operations when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

### 5. References

Abolhasan, Mehran. 2004. “A review of routing protocols for mobile ad hoc networks.” Ad Hoc Networks 2, no. 1 (January): 1-22. https://doi.org/10.1016/S1570-8705 (03) 00043-X.

Asare, Kennedy O., Yannik Terhorst, Julio Vega, Ella Peltonen, Eemil Lagerpetz, and Denzi Ferreira. 2021. “Predicting Depression From Smartphone Behavioral Markers Using Machine Learning Methods, Hyperparameter Optimization, and Feature Importance Analysis: Exploratory Study.” JMIR mHealth and uHealth 9, no. 7 (July). 10.2196/26540.

Ferreira, Denzil, Anind K. Dey, and Vassilis Kostakos. 2011. “Understanding Human-Smartphone Concerns: A Study of Battery Life.” Pervasive Computing, 19-33.

Williams, Brad, and Tracy Camp. 2002. “Comparison of broadcasting techniques for mobile ad hoc networks.” Proceedings of the 3rd ACM international symposium on Mobile ad hoc networking & computing, 194-205. https://doi.org/10.1145/513800.513825.

What is claimed is:

1. A computer-implemented method comprising:
using a computer system comprising a plurality of edge devices in a local computing environment, programming a first edge device of the plurality of edge devices to operate as an edge computing node, wherein the plurality of edge devices is configured to execute operations without requiring telecommunications to a remote server via an internet;
by the first edge device of the computer system, identifying one or more second edge devices of the plurality of edge devices being activated in the local computing environment;
by the first edge device of the computer system, adding the first edge device and the one or more second edge devices in an edge network;
by the first edge device of the computer system, retrieving, from the one or more second edge devices via the edge network, sensor data collected by the one or more second edge devices;
by the first edge device of the computer system, generating one or more data summaries from the sensor data based on one or more models;
by the first edge device of the computer system, storing the one or more data summaries in a storage associated with the first edge device; and
by the first edge device of the computer system, presenting the one or more data summaries via an application executing on the first edge device.

2. The computer-implemented method of claim 1, wherein identifying the one or more second edge devices is based on one or more of a network protocol, a proximity protocol, or a device discovery protocol.

3. The computer-implemented method of claim 1, further comprising, by the computer system, determining the first edge device to be the edge computing node using one or more voting protocols based on metadata associated with the plurality of edge devices, the metadata comprising one or more of a device type, a CPU type, or an amount of summary.

4. The computer-implemented method of claim 1, the edge network comprising a local mesh network.

5. The computer-implemented method of claim 1, wherein adding each of the first edge device and the one or more second edge devices in the edge network comprises one or more of:
supplying a session key for encrypted local communication;
providing one or more LAN access credentials;
providing one or more Wi-Fi router access credentials; or adding the one or more second edge devices to a list of devices in the edge network that is digitally stored at the edge computing node.

6. The computer-implemented method of claim 1, further comprising, by the first edge device of the computer system, transmitting the one or more data summaries or an update to a cloud computing system.

7. The computer-implemented method of claim 6, wherein transmitting the one or more data summaries or the update to the cloud computing system is based on a message bus or an event bus.

8. The computer-implemented method of claim 1, retrieving data from the one or more second edge devices via the edge network comprising:
by the first edge device of the computer system, determining a respective device type for each of the one or more second edge devices; and
by the first edge device of the computer system, transmitting, to each of the one or more second edge devices, a respective request for data formatted based on the respective device type, wherein the respective request for data is configured for causing the respective one of the one or more second edge devices to respond with the data requested.

9. The computer-implemented method of claim 8, wherein the respective request for the data comprises one or more of a single discrete command, a single discrete ping, or an application programming interface (API) call.

10. The computer-implemented method of claim 1, further comprising, by the first edge device of the computer system, de-identifying the one or more data summaries.

11. The computer-implemented method of claim 1, further comprising, by the first edge device of the computer system, encrypting the one or more data summaries.

12. The computer-implemented method of claim 1, wherein the storage is based on one or more of an in-app memory, an on-device memory, or a network-attached storage.

13. The computer-implemented method of claim 1, wherein the one or more data summaries comprise one or more mental health indicators comprising one or more of a sleeping pattern, a social activity, a mobility, or a daily routine.

14. The computer-implemented method of claim 1, further comprising, by the computer system, generating, based on the one or more data summaries, a personalized treatment via algorithmic interventions.

15. The computer-implemented method of claim 1, further comprising, by the computer system, generating, based on the one or more data summaries, an appraisal of mental health states and trends.

16. One or more non-transitory computer-readable storage media storing one or more sequences of instructions which, when executed using one or more processors, cause the one or more processors to execute:
using a computer system comprising a plurality of edge devices in a local computing environment, programming a first edge device of the plurality of edge devices to operate as an edge computing node, wherein the plurality of edge devices are configured to execute operations without requiring telecommunications to a remote server via an internet;
by the first edge device of the computer system, identifying one or more second edge devices of the plurality of edge devices being activated in the local computing environment;
by the first edge device of the computer system, adding the first edge device and the one or more second edge devices in an edge network;
by the first edge device of the computer system, retrieving, from the one or more second edge devices via the edge network, sensor data collected by the one or more second edge devices;
by the first edge device of the computer system, generating one or more data summaries from the sensor data based on one or more models;
by the first edge device of the computer system, storing the one or more data summaries in a storage associated with the first edge device; and
by the first edge device of the computer system, presenting the one or more data summaries via an application executing on the first edge device.

17. The one or more non-transitory computer-readable storage media of claim 16, further comprising sequences of instructions which, when executed using the one or more processors, cause the one or more processors to execute identifying the one or more second edge devices based on one or more of a network protocol, a proximity protocol, or a device discovery protocol.

18. The one or more non-transitory computer-readable storage media of claim 16, further comprising sequences of instructions which, when executed using the one or more processors, cause the one or more processors to execute determining the first edge device to be the edge computing node using one or more voting protocols based on metadata associated with the plurality of edge devices, the metadata comprising one or more of a device type, a CPU type, or an amount of summary.

19. The one or more non-transitory computer-readable storage media of claim 16, further comprising sequences of instructions which, when executed using the one or more processors, cause the one or more processors to execute adding each of the first edge device and the one or more second edge devices in the edge network using one or more of:
supplying a session key for encrypted local communication;
providing one or more LAN access credentials;
providing one or more Wi-Fi router access credentials; or
adding the one or more second edge devices to a list of devices in the edge network that is digitally stored at the edge computing node.

20. The one or more non-transitory computer-readable storage media of claim 16, further comprising sequences of instructions which, when executed using the one or more processors, cause the one or more processors to execute retrieving data from the one or more second edge devices via the edge network using:
determining a respective device type for each of the one or more second edge devices; and
transmitting, to each of the one or more second edge devices, a respective request for data formatted based on the respective device type, wherein the respective request for the data is configured for causing the respective one of the one or more second edge devices to respond with the data requested.

21. A computer system comprising a self-organizing edge system, the computer system comprising:
a cloud storage and analysis computer; and
an edge network that is communicatively coupled via an internetwork to the cloud storage and analysis computer, the edge network comprising an edge computing node, a plurality of edge devices, and an edge storage and analysis device;
wherein each edge device among the plurality of edge devices comprises one or more central processing units, one or more network interfaces that are configured to communicatively couple the one or more central processing units to a data communication network, and electronic digital random access memory storing one or more sequences of instructions which, when executed using the one or more central processing units, cause the one or more central processing units to execute:
programming a first edge device of the plurality of edge devices to operate as an edge computing node, wherein the plurality of edge devices are configured to execute operations without requiring telecommunications to a remote server via an internetwork;
by the first edge device of the computer system, identifying one or more second edge devices of the plurality of edge devices being activated in a local computing environment;
by the first edge device of the computer system, adding the first edge device and the one or more second edge devices in an edge network;
by the first edge device of the computer system, retrieving, from the one or more second edge devices via the edge network, sensor data collected by the one or more second edge devices;
by the first edge device of the computer system, generating one or more data summaries from the sensor data based on one or more models;
by the first edge device of the computer system, storing the one or more data summaries in a storage associated with the first edge device; and
by the first edge device of the computer system, presenting the one or more data summaries via an application executing on the first edge device.

22. The computer system of claim 21, further comprising sequences of instructions which, when executed using the one or more central processing units, cause the one or more central processing units to execute identifying the one or more second edge devices based on one or more of a network protocol, a proximity protocol, or a device discovery protocol.

23. The computer system of claim 21, further comprising sequences of instructions which, when executed using the one or more central processing units, cause the one or more central processing units to execute determining the first edge device to be the edge computing node using one or more voting protocols based on metadata associated with the plurality of edge devices, the metadata comprising one or more of a device type, a CPU type, or an amount of summary.

24. The computer system of claim 21, further comprising sequences of instructions which, when executed using the one or more central processing units, cause the one or more central processing units to execute adding each of the first edge device and the one or more second edge devices in the edge network using one or more of:
supplying a session key for encrypted local communication;
providing one or more LAN access credentials;
providing one or more Wi-Fi router access credentials; or
adding the one or more second edge devices to a list of devices in the edge network that is digitally stored at the edge computing node.

25. The computer system of claim 21, further comprising sequences of instructions which, when executed using the one or more central processing units, cause the one or more central processing units to execute retrieving data from the one or more second edge devices via the edge network using:
determining a respective device type for each of the one or more second edge devices; and
transmitting, to each of the one or more second edge devices, a respective request for data formatted based on the respective device type, wherein the respective request for data is configured for causing the respective second edge device to respond with the data requested.
